SEQUENCE LISTING (26 sequences)

SEQ ID NO: 1 - protein, 120 aa, Artificial Sequence
  anti-CEA VHH
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 2 - protein, 102 aa, Artificial Sequence
  anti-CD16 VHH
EVQLVESGGGLVQPGGSLRLSCSFPGSIFSLTMGWYRQAPGKERELVTSATPGGDTNYADFVKGRFTISRDNARSIIYLQMNSLKPEDTAVYYCYARTRNWG

SEQ ID NO: 3 - protein, 102 aa, Artificial Sequence
  anti-CD16 VHH
EVQLVESGGELVQAGGSLRLSCAASGLTFSSYNMGWFRRAPGKEREFVASITWSGRDTFYADSVKGRFTISRDNAKNTVYLQMSSLKPEDTAVYYCAANPWP

SEQ ID NO: 4 - protein, 102 aa, Artificial Sequence
  anti-CD16 VHH
EVQLVESGGGLVQPGESLTLSCVVAGSIFSFAMSWYRQAPGKERELVARIGSDDRVTYADSVKGRFTISRDNIKRTAGLQMNSLKPEDTAVYYCNAQTDLRD

SEQ ID NO: 5 - protein, 102 aa, Artificial Sequence
  anti-CD16 VHH
EVQLVESGGGLVQPGGSLTLSCVAAGSIFTFAMSWYRQAPRKERELVARIGTDDETMYKDSVKGRFTISRDNVKRTAGLQMNNLKPEDTAVYYCNARTDYRD

SEQ ID NO: 6 - protein, 123 aa, Artificial Sequence
  anti-Her2 sdAb
QVQLVQSGGGLVQAGGSLRLSCAASGRTFSSYAMAWFRQAPGKEREFVAAISWSGANIYVADSVKGRFTISRDNAKDTVYLQMNSLKPEDTAVYYCAVKLGFAPVEERQYDYWGQGTQVTVSS

SEQ ID NO: 7 - protein, 128 aa, Artificial Sequence
  anti-EGFR1 VHH
MAEVQQASGGGLVQAGGSLRLSCAASGRTETTSAIAWFRQAPGKEREFVAQISASGLGINYSGTVKGRFTISRDADKTTVYLQMNSLTPEDTAVYYCAAGFHYIAAIRRTTDFHFWGPGTLVTVSSGR

SEQ ID NO: 8 - protein, 121 aa, Artificial Sequence
  anti-F4+ETEC bacteria VHH
QVQLQESGGGLVQAGGSLRLSCEASGNVDRIDAMGWFRQAPGKQREFVGYISEGGILNYGDFVKGRFTISRDNAKNTVYLQMSNLKSEDTGVYFCAASHWGTLLIKGIEHWGKGTQVTVSS

SEQ ID NO: 9 - protein, 127 aa, Artificial Sequence
  anti-PS2-8 VHH
EVQLVESGGGLVQAGGSLRLSCAASGRSFSRDAMGWFRQAPGKERDVVAAINLNGGRTYSADSVKGRFTISRDNDKNTVYLQMSNLKPEDTAVYYCAAREGDVGLVSYKRSSNYPYWGQGTQVTVSS

SEQ ID NO: 10 - protein, 121 aa, Artificial Sequence
  anti-Huntavirus VHH
MAEVQLQASGGGLVQAGGSLRLSCAASGRTSSMYSMVWFRQAPGKEREFVAGIIWTSSLTYYADSLKGRFTISRDNAKNTVYLQMNSLKPEDTAIYYCAADTKTGGGGYEYWGQVTVTVSS

SEQ ID NO: 11 - protein, 119 aa, Artificial Sequence
  anti-Huntavirus VHH
MAEVQLQASGGGLVQPGGSLRLSCAASGSIFSSDVMGWFRQAPGKERELVAFITDDGGTNYADSVKGRFTISRDNAENTVSLQMNSLKPEDTAVYYCNARYYSGGYRNYWGQVTVTVSS

SEQ ID NO: 12 - protein, 115 aa, Artificial Sequence
  anti-CD16 VHH
EVQLVESGGGLVQPGGSLRLSCSFPGSIFSLTMGWYRQAPGKERELVTSATPGGDTNYADFVKGRFTISRDNARSIIYLQMNSLKPEDTAVYYCYARTRNWGTVWGQGTQVTVSS

SEQ ID NO: 13 - protein, 129 aa, Artificial Sequence
  anti-CD3 VHH
QVQLQESGGGLVQAGGSLRLSCAASGRTFSNYHMGWFRQAPGKERELVAAISGSGGSTYYTDSVKGRFTISRNNAKNTMSLQMSNLKPEDTGVYYCTTPTEKGSSIDYWGQGTQVTVSSGRYPYDVPDY

SEQ ID NO: 14 - DNA, 654 bp, Artificial Sequence
  anti-CD19Fab light chain-nucleic acid
  VL (1)..(336)
  CL (337)..(654)
gatattcagatgactcagagcccttcctcactgtccgccagcgtcggggatcgggtcactattacatgcaaagcaagccagagcgtcgactatgatggggactcctatctgaactggtaccagcagaagccaggcaaagctcccaagcggctgatctacgacgcatcaaatctggtgagcggcgtcccttccagattctctgggagtggatcaggcacagattttaccctgacaattagctccctgcagcctgaggacttcgccacttactattgccagcagagcacagaagatccatggacttttggcgggggaaccaaagtggagatcaagagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 15 - protein, 218 aa, Artificial Sequence
  anti-CD19Fab light chain - amino acid
  VL (1)..(109)
  CL (110)..(218)
DIQMTQSPSSLSASVGDRVTITCKASQSVDYDGDSYLNWYQQKPGKAPKRLIYDASNLVSGVPSRFSGSGSGTDFTLTISSLQPEDFATYYCQQSTEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - DNA, 666 bp, Artificial Sequence
  anti-CD19Fab heavy chain-nucleic acid
  VH (1)..(372)
  CH (373)..(666)
caggtgcagctggtccagtccggggcagaagtgaagaaaccaggagcctctgtgaaagtcagttgtaaggcttcaggctataccttcatctcttactggatgaactgggtgagacaggcaccaggacagggactggagtggatgggacagatttggcctggcgatggggacaccaactataatcagctgctgaaggataaagcaactctgaccgccgacaagagcgcctccaccgcttacatggagctgtctagtctgcgaagcgaagacacagctgtgtactattgcgcacggagagaaaccacaactgtgggccgctactattacgcaatggattactggggacagggcacactggtgactgtctcaagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtt

SEQ ID NO: 17 - protein, 222 aa, Artificial Sequence
  4. anti-CD19Fab heavy chain-amino acid
  VH (1)..(127)
  CH (128)..(222)
QVQLVQSGAEVKKPGASVKVSCKASGYTFISYWMNWVRQAPGQGLEWMGQIWPGDGDTNYNQLLKDKATLTADKSASTAYMELSSLRSEDTAVYYCARRETTTVGRYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 18 - DNA, 639 bp, Artificial Sequence
  anti-CD20Fab light chain-nucleic acid
  VL (1)..(321)
  CL (322)..(639)
cagatcgtgctgagccagagccccgccatcctgagcgccagccccggcgagaaggtgaccatgacctgcagggccagcagcagcgtgagctacatccactggttccagcagaagcccggcagcagccccaagccctggatctacgccaccagcaacctggccagcggcgtgcccgtgaggttcagcggcagcggcagcggcaccagctacagcctgaccatcagcagggtggaggccgaggacgccgccacctactactgccagcagtggaccagcaacccccccaccttcggcggcggcaccaagctggagatcaagaggacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 19 - protein, 213 aa, Artificial Sequence
  anti-CD20Fab light chain-amino acid
  VL (1)..(107)
  CL (108)..(213)
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - DNA, 654 bp, Artificial Sequence
  anti-CD20Fab heavy chain-nucleic acid
  VH (1)..(360)
  CH (361)..(654)
caggtgcagctgcagcagcccggcgccgagctggtgaagcccggcgccagcgtgaagatgagctgcaaggccagcggctacaccttcaccagctacaacatgcactgggtgaagcagacccccggcaggggcctggagtggatcggcgccatctaccccggcaacggcgacaccagctacaaccagaagttcaagggcaaggccaccctgaccgccgacaagagcagcagcaccgcctacatgcagctgagcagcctgaccagcgaggacagcgccgtgtactactgcgccaggagcacctactacggcggcgactggtacttcaacgtgtggggcgccggcaccaccgtgaccgtgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtt

SEQ ID NO: 21 - protein, 218 aa, Artificial Sequence
  anti-CD20Fab heavy chain-amino acid
  VH (1)..(122)
  CH (123)..(218)
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 22 - DNA, 639 bp, Artificial Sequence
  anti-Her2Fab light chain-nucleic acid
  VL (1)..(321)
  CL (321)..(639)
gatattcagatgacccagagcccgagcagcctgtccgcaagcgtcggcgaccgcgtcacgattacctgccgtgcaagccaggatgtcaacaccgcagtggcttggtatcagcaaaaaccgggtaaagcgccgaaactgctgatttatagtgcctcctttctgtactctggcgtgccgagtcgtttctcaggttcgcgcagcggcaccgattttaccctgacgatcagctctctgcagccggaagacttcgccacgtattactgccagcaacattataccacgccgccgacctttggtcaaggcacgaaagtcgaaattaaaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 23 - protein, 213 aa, Artificial Sequence
  anti-Her2Fab light chain-amino acid
  VL (1)..(107)
  CL (108)..(213)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - DNA, 651 bp, Artificial Sequence
  anti-Her2Fab heavy chain-nucleic acid
  VH (1)..(357)
  CH (358)..(651)
gaagtgcagctggttgaatctggcggtggcctggttcaaccgggtggctccctgcgtctgtcatgtgcagcatcgggtttcaacattaaagatacctatatccactgggttcgtcaggcaccgggtaaaggcctggaatgggtcgctcgcatttacccgaccaatggttatacgcgttacgcggatagcgtgaaaggccgcttcaccatcagcgcagacacctctaaaaacacggcttatctgcagatgaactcgctgcgtgcggaagatacggccgtttattactgtagccgctggggtggcgacggcttttacgcaatggattactggggtcaaggcacgctggtcacggtctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtt

SEQ ID NO: 25 - protein, 217 aa, Artificial Sequence
  anti-Her2Fab heavy chain-amino acid
  VH (1)..(119)
  CH (120)..(217)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 26 - protein, 6 aa, Artificial Sequence
  linker with two C-terminal cycterine
CGGGGC

(19) United States
(12) Patent Application Publication
(10) Pub. No.: US 2021/0032362 A1
(43) Pub. Date: Feb. 4, 2021

(54) BISPECIFIC ANTIBODY WITH PROLONGED HALF-LIFE AND ENHANCED ANTI-TUMOR EFFECT

(71) Applicant: SOUND BIOPHARMACEUTICALS CO. LTD., Chengdu (CN)

(72) Inventor: Fanxin MA, Chengdu (CN)

(21) Appl. No.: 17/046,671

(22) PCT Filed: Apr. 3, 2019
(86) PCT No.: PCT/CN2019/081164

§ 371 Date: Oct. 9, 2020

(30) Foreign Application Priority Data
Apr. 9, 2018 (CN) 201810310970.X

(51) Int. Cl. C07K 16/30; C07K 16/28; A61P 35/00

(52) U.S. Cl. CPC C07K 16/3007; C07K 16/2809; A61P 35/00; C07K 2317/31; C07K 2317/55; A61K 2039/505; C07K 2317/51; C07K 2317/515; C07K 2317/73; C07K 2317/94; C07K 2317/569

(57) ABSTRACT

Disclosed is a PEGylated bispecific antibody, comprising (a) an antigen-binding fragment (Fab), which has a light chain variable region (VL) and a light chain constant region (CL), a heavy chain Variable region (VH) and a part of the heavy chain constant region (CH1); (b) a single domain antigen binding fragment (VHH) fused to the C-terminus of the part of the heavy chain constant region (CH1) of the antigen binding fragment (Fab), and (c) Polyethylene glycol (PEG) fused to the C-terminus of the light chain constant region (CL) of the antigen-binding fragment (Fab). The PEGylated S-Fab (PEG-S-Fab) retained the binding specificity to both tumor cells and T cells. PEG-S-Fab enhanced the plasma stability and had a 12-fold increased half-life of S-Fab. PEG-S-Fab also had more potent tumor inhibiting efficacy in xenograft mouse model. Those data suggest that PEGylation can be an effective approach to enhance anti-tumor properties of bispecific antibodies.

### TECHNICAL FIELD

The present invention relates to bispecific antibodies, and in particular to PEGylated bispecific antibodies with prolonged half-life and enhanced anti-tumor effect.

### BACKGROUND

Bispecific antibody has been developed as a powerful approach in cancer immunotherapy by engaging immune cells directly to the cancer cells. Bispecific antibodies have two different antigen binding sites with one recognizing the tumor cells and the other recognizing the immune cells, usually T cells or natural killer (NK) cells. Various bispecific antibody formats have been studied, including IgG based full-length formats (such as the complete bispecific antibody), single chain-based formats (including tandem single-chain variable fragments (ScFv)) and bispecific T-cell engager (BiTE).

In order to enhance the tumor tissue penetration of full-size IgG antibodies, and improve the stability of ScFv and BiTE, the bispecific single domain antibody-linked Fab (S-Fab) format was developed, which can bind to diverse epitopes and use prokaryotic expression systems. Derived from the natural camel heavy-chain only antibodies, single domain antibodies lack the first constant (CH1) domain and light chain, and are consequently referred to as nanobodies or VHHs. Nanobodies are small-sized and generally more stable than conventional scFv or BiTE, making them good specialty for constructing bispecific antibodies. In a previous study, a bispecfic S-Fab antibody was constructed by linking the single-domain nanobody anti-CEA to an CD3-Fab. The S-Fab exhibited excellent tumor cell killing activities in vitro. (L. Li, P. He, C. Zhou, L. Jing, B. Dong, S. Chen, N. Zhang, Y. Liu, J. Miao, Z. Wang, Q. Li, A novel bispecific antibody, S-Fab, induces potent cancer cell killing, Journal of immunotherapy, 38 (2015) 350-356).

However, Fab fragments (including S-Fab) still have short plasma half-lives as a result of rapid degradation in vivo, due to the lack of the constant regions (Fc), which makes them less ideal for long-term clinical use.

### SUMMARY

In one aspect, provided is a PEGylated bispecific antibody (also called PEG-S-Fab herein), comprising (a) an antigen-binding fragment (Fab) having a light chain variable region (VL) and a light chain constant region (CL), as well as a heavy chain variable region (VH) and a part of a heavy chain constant region (CH1); (b) a single domain antigen-binding fragment (VHH) fused to the C-terminus of the part of the heavy chain constant region (CH1) of the antigen binding fragment (Fab); and (c) polyethylene glycol (PEG) fused to the C-terminus of the light chain constant region (CL) of the antigen-binding fragment (Fab). The molecular weight of PEG suitable for use in the present disclosure can be determined according to the routine test in the field. In some embodiments, the molecular weight of the polyethylene glycol is 10,000 to 30,000. In another embodiments, the molecular weight of the polyethylene glycol is 20,000.

The present invention also provides a pharmaceutical composition comprising the PEGylated bispecific antibody, and a pharmaceutically acceptable carrier or excipient. Correspondingly, the present invention provides a host cell comprising one or more polynucleotides encoding the bispecific antibody.

In another aspect, provided is an engineered bispecific antibody comprising (a) an antigen-binding fragment (Fab) having a light chain variable region (VL) and a light chain constant region (CL), a heavy chain variable region (VH) and a part of a heavy chain constant region (CH1); and (b) a single domain antigen binding fragment (VHH) fused to the C-terminus of the part of the heavy chain constant region (CH1) of the antigen binding fragment (Fab); wherein the C-terminus of the light chain constant region (CL) of the antigen-binding fragment (Fab) is engineered to have at least one cysteine residue. Correspondingly, the present invention provides a use of the engineered bispecific antibody in preparing the PEGylated bispecific antibody of the present invention.

Another aspect of the present invention provides a method of treating cancer, comprising contacting the PEGylated bispecific antibody of the present invention with cancer cells. Accordingly, the present invention provides a method of treating cancer in a subject, which comprises administering to the subject a therapeutically effective amount of the PEGylated bispecific antibody of the present invention.

Another aspect of the present invention provides a host cell comprising one or more polynucleotides encoding the bispecific antibody as described above. In some embodiments, the host cell is E. coli. The manipulation of polynucleotides involves knowledge and experimental operations in the fields of molecular biology, genetic engineering, protein engineering, etc., which are well known to those skilled in the art.

The inventors explored a thiol site-specific PEGylation to improve the half-life (a2) of S-Fab bispecific antibody. In this study, a functionalized 20 kDa linear methoxy PEG maleimide (MAL-PEG-OMe) was used to conjugate S-Fab. The PEGylated S-Fab (PEG-S-Fab) retained the binding specificity to both tumor cells and T cells. PEG-S-Fab enhanced the plasma stability and had a 12-fold increased half-life of S-Fab. PEG-S-Fab also had more potent tumor inhibiting efficacy in xenograft mouse model. Those data suggest that PEGylation can be an effective approach to enhance anti-tumor properties of bispecific antibodies.

### BRIEF DESCRIPTION OF DRAWINGS

FIG. 1 Expression and purification of S-Fab from E. coli. A, The bacterial S-Fab expression constructs contain a pelB signal sequence, an anti-CD3 (human UTCH1 clone) VH (or VL) and CH (CL), and an anti-CEA-VHH. To facilitate antibody detection and purification, a flag-tag and a his6-tag were added to the C-terminal end of heavy and light chain, respectively; B, Schematic presentation of S-Fab after co-expression; C, Coomassie blue-stained SDS-PAGE of the purified S-Fab after the two-step purification; D, Gel filtration analysis showed that the molecular weight of S-Fab was approximately 130 kD.

FIG. 2 PEGylation of bispecific S-Fab using 20 kDa linear methoxy PEG maleimide (MAL-PEG-OMe). S-Fab reacted with the functionalized PEG at a series of ratios (0:1, 10:1, 20:1, 40:1 and 60:1) mol equiv of PEG:S-Fab and shaking at 22° C. for 2 hrs, the samples were then examined with SDS-PAGE (5 ul/sample). A. Coomassie blue staining for the S-Fab with different molar ratio of PEG:S-Fab in PEGylation process. B. Barium iodide complex dye staining for the S-Fab with different molar ratios of PEG:S-Fab. C. Western blot using anti-flag antibody to detect the VH—CH-CEA chain. D. Western blot using anti-his6 antibody to detect the VL-CL chain. Note: 0, 10, 20, 40 and 60 represent as 0:1, 10:1, 20:1, 40:1 and 60:1 for the molar ratio of PEG:Fab.

FIG. 3 Purification of PEG-S-Fab. The gel filtration was used to fraction the PEGylation mixture (Fractions 1, 2, 3). A. Chromatogram of PEG-S-Fab. B. Coomassie blue staining for the fractions of PEG-S-Fab purification. C. Barium iodide complex staining for the fractions of PEG-S-Fab purification.

FIG. 4 PEG-S-Fab can bind CEA on tumor cells and CD3⁺ on T cells. Flow cytometry analysis with PEG-S-Fab and S-Fab was performed on CEA-positive LS174T cells (A), CEA-negative SKOV3 cells (B), and CD3⁺ T cells (C). Positive control anti-CD3 antibody OKT3 was used for T cell flow cytometry. Confocal microcopy of immunofluorescence staining of S-Fab (D) and PEG-S-Fab (E) in LS174T cells (upper panel) and SKOV3 cells (lower panel), respectively. The bar scale represents 30 μm.

FIG. 5 PEG-S-Fab has potent specific cytotoxicity against tumor cells. A. The cytotoxic assay using CEA-positive LS174T cells. B. The cytotoxic assay using CEA-negative SKOV3 cells. The cytotoxic assays were performed as described in section 2.8. Different concentrations of S-Fab or PEG-S-Fab were incubated with tumor cells and effector T cells (E/T=10). All data are showed as the mean of triplicates, with error bars representing the SD.

FIG. 6 Pharmacokinetic and stability analysis of serum concentrations of PEG-S-Fab and S-Fab. A. Standard curve for the quantified ELISA. B. The serum protein concentration-time curve after intravenous administration. (Note: Each data point was expressed as the mean±SEM). C-D. Western blotting to detect the resulting S-Fab (C) and PEG-S-Fab (D) using anti-flag antibody. E-F. Western blotting to detect the resulting S-Fab (C) and PEG-S-Fab (D) using anti-his antibody. (Note: M, P—S—Fab and P represent as marker, PEG-S-Fab and plasma only, respectively).

FIG. 7 PEGylation of S-Fab induces more potent in vivo anti-tumor activity. B-NDG mice (n=6 per group) were subcutaneously engrafted with LS174T cells and human PBMCs. The mice were then treated with PBS alone, 0.3 nmol of S-Fab or S-Fab-PEG20K daily over six days. The data represent the average tumor volume of six mice. The error bars represent the SEM. Data were analyzed with two-way ANOVA analysis using GraphPad Prism 5 software, and *** represents as p<0.01 when compared the vehicle with S-Fab or PEG-S-Fab groups.

### DETAILED DESCRIPTION OF THE INVENTION

### Definition

It should be noted that the term “a” or “an” entity refers to one or more of that entity, for example, “a bispecific antibody” is understood to represent one or more bispecific antibodies. Likewise, the terms “a”, “one or more” and “at least one” are used interchangeably herein.

The term “antibody” or “antigen-binding polypeptide” as used herein refers to a polypeptide or polypeptide complex that specifically recognizes and binds to one or more antigens. Antibodies are complete antibodies or any antigen-binding fragments or single chains thereof. Therefore, the term “antibody” includes any protein or peptide that contains at least a part of an immunoglobulin molecule that has the biological activity of binding to an antigen. Such examples include, but are not limited to, the complementarity determining region (CDR) of the heavy/light chain or its ligand binding portion, the variable region of the heavy or light chain, the constant region of the heavy or light chain, and the framework (FR) region or any part thereof, or at least part of a binding protein. The term antibody also encompasses polypeptides or polypeptide complexes that have antigen-binding ability once activated. In some examples, for example, certain immunoglobulin molecules derived from or based on the engineering of camelid immunoglobulins. The whole immunoglobulin molecule may only consist of heavy chains without light chains, see for example Hamers-Casterman et al., Nature 363:446-448 (1993).

The term “specific binding” or “specific to” generally means that the antibody binds to the epitope through its antigen binding domain, and the binding requires a certain complementarity between the antigen binding domain and the epitope. According to this definition, when an antibody can bind to a specific epitope more rapidly via its antigen binding domain than to a random unrelated epitope, the antibody is said to “specifically bind” to that epitope. The term “specificity” is used to quantify the relative affinity of a specific antibody to a specific epitope. For example, for a given epitope, antibody “A” is considered to have a higher specificity than antibody “B”, or antibody “A” binds to epitope “C” more specifically than it binds to related epitope “D”.

The term “treatment” as used herein refers to a therapeutic or preventive approach in which a subject is prevented from or slowed down (alleviated) an undesirable pathological change or disorder, such as the progression of cancer. Beneficial or desired clinical results include, but are not limited to, alleviation of symptoms, reduction of disease severity, stabilization of the disease state (that is, no deterioration), delay or slowing of disease progression, improvement or alleviation of disease state, and alleviation (either locally or systematically), detectable or undetectable. The term “treatment” also refers to prolonged survival compared to expected survival if not receiving the treatment. Subject in need of treatment include those who already have a disease or condition, as well as those who tend to have the disease or condition or those who need to prevent the disease or condition.

“Subject” or “individual” or “animal” or “patient” or “mammal” refers to any subject for which diagnosis, prognosis, or treatment is desired, especially a mammalian subject. Mammal subjects include humans, domestic animals, farm animals, zoo animals, sports animals or pets, such as dogs, cats, guinea pigs, rabbits, rats, mice, horses, bovines, cows, and the like.

The term “patient in need of treatment” or “subject in need of treatment” includes subjects who can benefit from the administration of the antibody or composition of the present invention, such as mammalian subjects, for purposes of such as detection, diagnostic procedures and/or treatment.

As confirmed in the examples, an exemplary bispecific antibody is an antibody that targets two different antigens, one of which is present on tumor cells or microorganisms, and the other is on immune cells. When administered to an individual, the bispecific antibody specifically binds to tumor cells or microorganisms, and at the same time specifically binds to immune cells (such as cytotoxic cells). This dual binding can cause the bound tumor or microorganism to be killed by the host's immune system.

A “single domain antigen-binding fragment” or “single domain antibody fragment” or “VHH” is an antigen-binding fragment capable of binding to an antigen without being equipped with a light chain. VHH was originally isolated from a single domain antibody (sdAb) as a single antigen-binding fragment. The first known single domain antibody was isolated from camel (Hamers-Casterman et al., Nature 363:446-8 (1993)) and later from cartilaginous fish. Camels produce functional antibodies without light chains, and their single N-terminal domain (VHH) binds to antigen without domain pairing (see Harmsen and Haard, App Microbiol Biotechnol, 77:13-22 (2007) for review). Single domain antibodies do not include the CH1 domain which, in conventional antibodies, interacts with the light chain.

VHH contains four framework regions (FR1-FR4) constituting the core structure of an immunoglobulin domain and three complementarity determining regions (CDR1-CDR3) involved in antigen binding. Compared with the human VH domain, the VHH framework region shows high sequence homology (>80%) with the human VH domain. See Harmsen and Haard, 2007, which further describes: “the most characteristic feature of VHH lies in the amino acid substitutions at the four FR2 positions (positions 37, 44, 45 and 47; Kabat numbering), which are conserved and involved in hydrophobic interactions with the VL domain in conventional VH structures”. VHH usually has different amino acids at these and other positions that are highly conserved in conventional VH (such as LeulISer, Val37Phe or Tyr, Gly44Glu, Leu45Arg or Cys, Trp47Gly).

Harmsen and Haard, 2007 also described: VHH CDR has some known characteristic features. For example, the N-terminal part of CDR1 is more variable than conventional antibodies. Moreover, certain VHHs have a next ended CDR3 which is usually stabilized by forming additional disulfide bonds with the cysteine in CDR1 or FR2, resulting in the CDR3 loop folding over the entire interface of the previous VL. The specific subfamily of llama VHH (VHH3) also contains an extended CDR3 which is stabilized by forming an additional disulfide bond width cysteine at position 50 of FR2.

Many single domain antibodies (sdAbs) are known in the art and can be easily prepared from animals such as camels. Based on these sdAbs, their VHH can be easily identified and prepared. Table 1 lists many non-limiting examples of VHH and sdAb. Therefore, in some embodiments, the present invention provides a polypeptide comprising each such disclosed sequence or its equivalent and a polynucleotide encoding each polypeptide.

TABLE 1
Exemplary single domain antigen binding fragments (VHH)
and single domain antibodies (sdAb)

1. anti-CEA VHH (SEQ ID NO: 1)
EVQLVESGGG FVQAGESLTL SCTSSTLTFT PYRMAWYRQA PGKQRDLVAD ISSGDGRTTN
YADFAKGRFT ISRDNIKNTV FLRMTNLKPE DTAVYYCNTF VSFVGIARSW GQGTQVTVSS
2. anti-CD16 VHH (SEQ. ID NO: 2)
EVQLVESGGG LVQPGGSLRL SCSFPGSIFS LTMGWYRQAP GKERELVTSA TEGGDTNYAD
FVKGRFTISR DNARSIIYLQ MNSLKPEDTA VYYCYARTRN WG
3. anti-CD16 VHH (SEQ ID NO: 3)
EVQLVESGGE LVQAGGSLRL SCAASGLTFS SYNMGWFRRA PGKEREFVAS ITWSGRDTFY
ADSVKGRFTI SRDNAKNTVY LQMSSLKPED TAVYYCAANP WP
4. anti-CD16 VHH (SEQ ID NO: 4)
EVQLVESGGG LVQEGESLTL SCVVAGSIFS FAMSWYRQAP GKERELVARI GSDDRVTYAD
SVKGRFTISR DNIKRTAGLQ MNSLKPEDTA VYYCNAQTDL RD
5. anti-CD16 VHH (SEQ ID NO: 5)
EVQLVESGGG LVQPGGSLTL SCVAAGSIFT FAMSWYRQAP RKERELVARI GTDDETMYKD
SVKGRFTISR DNVKRTAGLQ MNNLKPEDTA VYYCNARTDY RD
6. anti-Her2 sdAb (SEQ ID NO: 6)
QVQLVQSGGG LVQAGGSLRL SCAASGRTFS SYAMAWFRQA PGKEREFVAA ISWSGANIYV
ADSVKGRFTI SRDNAKDTVY LQMNSLKPED TAVYYCAVKL GFAPVEERQY DYWGQGTQVT
VSS
7. anti-EGFR1 VHH (SEQ ID NO: 7)
MAEVQQASGG GLVQAGGSLR LSCAASGRTE TTSAIAWFRQ APGKEREFVA QISASGLGIN
YSGTVKGRFT ISRDADKTTV YLQMNSLTPE DTAVYYCAAG FHYIAAIRRT TDFHFWGPGT
LVTVSSGR
8. anti-F4 + ETEC bacteria VHH (SEQ ID NO: 8)
QVQLQESGGG LVQAGGSLRL SCEASGNVDR IDAMGWERQA PGKQREFVGY ISEGGILNYG
DEVKGRETIS RDNAKNTVYL QMSNLKSEDT GVYFCAASHW GTLLIKGTEH WGKGTQVTVS
S
9. anti-PS2-8 VHH (SEQ ID NO: 9)
EVQLVESGGG LVQAGGSLRL SCAASGRSFS RDAMGWFRQA PGKERDVVAA INLNGGRTYS
ADSVKGPFTI SRDNDKNTVY LQMSNLKPED TAVYYCAARE GDVGLVSYKR SSNYPYWGQG
TQVTVSS
10. anti-Huntavirus VHH (SEQ ID NO: 10)
MAEVQLQASG GGLVQAGGSL RLSCAASGRT SSMYSMVWFR QAPGKEREFV AGIIWTSSLT
YYADSLKGRF TISRDNAKNT VYLQMNSLKP EDTAIYYCAA DTKTGGGGYE YWGQVTVTVS
S
11. anti-Huntavirus VHH (SEQ ID NO: 11)
MAEVQLQASG GGLVQPGGSL RLSCAASGSI FSSDVMGWFR QAPGKERELV AFITDDGGTN
YADSVKGRFT ISRDNAENTV SLQMNSLKPE DTAVYYCNAR YYSGGYRNYW GQVTVTVSS
12. anti-CD16 VHH (SEQ ID NO: 12)
EVQLVESGGG LVQPGGSLRL SCSFPGSIFS LTMGWYRQAP GKERELVTSA TPGGDTNYAD
FVKGRETISR DNARSIIYLQ MNSLKPEDTA VYYCYARTPN WGTVWGQGTQVTVSS
13. anti-CD3 VHH (SEQ ID NO: 13)
GVQLQESGGG LVQAGGSLRL SCAASGRTFS NYHMGWFRQA PGKERELVAA ISGSGGSTYY
TDSVFGRFTI SPNNAKNTMS LQMSNLKPED TGVYYCTTPT EKGSSIDYWG QGTQVTVSSG
RYPYDVPDY

In some embodiments, the Fab or VHH (or VHH′) fragment of the bispecific antibody has immunologic specificity to tumor antigens.

“Tumor antigen” is anantigenic substance produced in tumor cells, that is, it causes an immune response from the host. Tumor antigens can be used to identify, tumor cells and are potential candidates for cancer treatment. The normal protein in the body is not antigenic. However, certain proteins are produced or overexpressed during the tumorigenesis process and therefore appear to be “foreign” to the body. This can include normal proteins that well evade the immune system, proteins that are usually produced in very small amount, proteins that are usually only produced at specific developmental stages, or proteins whose structure is modified due to mutations.

Many tumor antigens are known in the art, and many new tumor antigens can be easily discovered through screening. Non-limiting examples of tumor antigens include EGFR, Her2, EpCAM, CD20, CD30, CD33, CD47, CD52, CD 133, CEA, gpA33, mucin, TAG-72, CIX PSMA, folate binding protein, GD2, GD3, GM2, VEGF, VEGFR, Integrin, αVβ3, α5β1, ERBB2, ERBB3, MET, IGF1R, EPHA3, TRAILR1, TRAILR2, RANKL, FAP and Tenascin.

In some aspects, Fab or VHH fragments have specificity for proteins that are overexpressed on tumor cells compared to corresponding non-tumor cells. “Corresponding non-tumor cells” refers to non-tumor cells that have the same cell type as the cells from which the tumor cells are derived. It should be noted that such proteins are not necessarily different from tumor antigens. Non-limiting examples include carcinoembryonic antigen (CEA), which is overexpressed in most colon, rectal, breast, lung, pancreatic, and gastrointestinal cancers; mygulin receptor (HER-2, neu or c-erbB-2), which is usually overexpressed in breast, ovarian, rectal, lung, prostate and cervical cancers; epidermal growth factor receptor, which is overexpressed in a series of solid tumors (including breast cancer, head and neck cancer, non-small cell lung cancer and prostate cancer); asialoglycoprotein receptor; transferin receptor; serine protease inhibitor enzyme complex receptor expressed on liver cells; fibroblast growth factor receptor (FGFR) over-expressed on pancreatic ductal adenocarcinoma cells; vascular endothelial growth factor receptor (VEGFR) for anti-angiogenesis gene therapy; folate receptor selectively overexpressed in 90% of non-mucinous ovarian cancer; polysaccharide-protein complexes (glycocalyx) on cell surface; carbohydrate receptors; and polyimmunoglobulin receptors, which are beneficial for gene transfer to respiratory epithelial cells and are attractive for the treatment of lung diseases (such as cystic fibrosis).

In some aspects, the Fab portion includes: one or two amino acid sequences selected from SEQ ID NO: 14-25 (Table 2), or optionally with one or two or three insertions, deletions or substitutions.

TABLE 2
Examples of Fab sequences

1. anti-CD19Fab light chain-nucleic acid (SEQ ID NO: 14)
GATATTCAGATGACTCAGAGCCCTTCCTCACTGTCCGCCAGCGTCGGGGATCGGGTCACT
ATTACATGCAAAGCAAGCCAGAGCGTCGACTATGATGGGGACTCCTATCTGAACTGGTAC
CAGCAGAAGCCAGGCAAAGCTCCCAAGCGGCTGATCTACGACGCATCAAATCTGGTGAGC
GGCGTCCCTTCCAGATTCTCTGGGAGTGGATCAGGCACAGATTTTACCCTGACAATTAGC
TCCCTGCAGCCTGAGGACTTCGCCACTTACTATTGCCAGCAGAGCACAGAAGATCCATGG
ACTTTTGGCGGGGGAACCAAAGTGGAGATCAAGAGA (VL)
ACGGTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGC
AAGGACAGCACCTACAGCCTCAGCAGCACCCGACGGCTGAGCAAAGCAGACTACGAGAAA
CACAAAGTCTACGCCTGCGAAGTCACCCATCAGGGCCTGAGCTCGCCCGTCACAAAGAGC
TTCAACAGGGGAGAGTGT (CL)
2. anti-CD19Fab light chain - amino acid (SEQ ID NO: 15)
DIQMTQSPSSLSASVGDRVTITCKASQSVDYDGDSYLNWYQQKPGKAPKRLIYDASNLVS
GVPSRFSGSGSGTDFTLTISSLQPEDFATYYCQQSTEDPWTFGGGTKVEIKR (VL)
TVAAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC (CL)
3. anti-CD19Fab heavy chain-nucleic acid (SEQ ID NO: 16)
CAGGTGCAGCTGGTCCAGTCCGGGGCAGAAGTGAAGAAACCAGGAGCCTCTGTGAAAGTC
AGTTGTAAGGCTTCAGGCTATACCTTCATCTCTTACTGGATGAACTGGGTGAGACAGGCA
CCAGGACAGGGACTGGAGTGGATGGGACAGATTTGGCCTGGCGATGGGGACACCAACTAT
AATCAGCTGCTGAAGGATAAAGCAACTCTGACCGCCGACAAGAGCGCCTCCACCGCTTAC
ATGGAGCTGTCTAGTCTGCGAAGCGAAGACACAGCTGTGTACTATTGCGCACGGAGAGAA
ACCACAACTGTGGGCCGCTACTATTACGCAATGGATTACTGGGGACAGGGCACACTGGTG
ACTGTCTCAAGC (VH)
GCTAGCACCAAGGGCCCATCGGTCTTCCCCCTGGCACCCTCCTCCAAGAGCACCTCTGGG
GGCACAGCGGCCCTGGGCTGCCTGGTCAAGGACTACTTCCCCGAACCGGTGACGGTGTCG
TGGAACTCAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCACCCAGACC
TACATCTGCAACGTGAATCACAAGCCCAGCAACACCAAGGTGGACAAGAAAGTT (CH)
4. anti-CD19Fab heavy chain-amino acid (SEQ ID NO: 17)
QVQLVQSGAEVKKPGASVKVSCKASGYTFISYWMNWVRQAPGQGLEWMGQIWPGDGDTNY
NQLLKDKATLTADKSASTAYMELSSLRSEDTAVYYCARPETTTVGRYYYAMDYWGQGTLV
TVSS (VH)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKV (CH)
5. anti-CD20Fab light chain-nucleic acid (SEQ ID NO: 18)
CAGATCGTGCTGAGCCAGAGCCCCGCCATCCTGAGCGCCAGCCCCGGCGAGAAGGTGACC
ATGACCTGCAGGGCCAGCAGCAGCGTGAGCTACATCCACTGGTTCCAGCAGAAGCCCGGC
AGCAGCCCCAAGCCCTGGATCTACGCCACCAGCAACCTGGCCAGCGGCGTGCCCGTGAGG
TTCAGCGGCAGCGGCAGCGGCACCAGCTACAGCCTGACCATCAGCAGGGTGGAGGCCGAG
GACGCCGCCACCTACTACTGCCAGCAGTGGACCAGCAACCCCCCCACCTTCGGCGGCGGC
ACCAAGCTGGAGATCAAGAGG (VL)
ACGGTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGC
AAGGACAGCACCTACAGCCTCAGCAGCACCCTGAGGCTGAGCAAAGCAGACTACGAGAAA
CACAAAGTCTACGCCTGCGAAGTCACCCATCAGGGCCTGAGCTCGCCCGTCACAAAGAGC
TTCAACAGGGGAGAGTGT (CL)
6. anti-CD20Fab light chain-amino acid (SEQ ID NO: 19)
QIVLSQSPAILSASPGEKVTMTCRASSSVSYIHWFQQKPGSSPKPWIYATSNLASGVPVR
FSGSGSGTSYSLTISRVEAEDAATYYCQQWTSNPPTFGGGTKLEIKR (VL)
TVAAPSVFIF PPSDEQLKSGTASVVCLLNNFYPREAKVQWKVDNALQGNSQESVTEQDS
KDSTYSLSSTLTLSKADYEKHKVYACEVTHQGLSSPVTKSFNRGEC (CL)
7. anti-CD20Fab heavy chain-nucleic acid (SEQ ID NO: 20)
CAGGTGCAGCTGCAGCAGCCCGGCGCCGAGCTGGTGAAGCCCGGCGCCAGCGTGAAGATG
AGCTGCAAGGCCAGCGGCTACACCTTCACCAGCTACAACATGCACTGGGTGAAGCAGACC
CCCGGCAGGGGCCTGGAGTGGATCGGCGCCATCTACCCCGGCAACGGCGACACCAGCTAC
AACCAGAAGTTCAAGGGCAAGGCCACCCTGACCGCCGACAAGAGCAGCAGCACCGCCTAC
ATGCAGCTGAGCAGCCTGAGCAGCGAGGACAGCGCCGTGTACTACTGCGCCAGGAGCACC
TACTACGGCGGCGACTGGTACTTCAACGTGTGGGGCGCCGGCACCACCGTGACCGTGAGC (VH)
GCTAGCACCAAGGGCCCATCGGTCTTCCCCCTGGCACCCTCCTCCAAGAGCACCTCTGGG
GGCACAGCGGCCCTGGGCTGCCTGGTCAAGGACTACTTCCCCGAACCGGTGACGGTGTCG
TGGAACTCAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCACCCAGACC
TACATCTGCAACGTGAATCACAAGCCCAGCAACACCAAGGTGGACAAGAAAGTT (CH)
8. anti-CD20Fab heavy chain-amino acid (SEQ ID NO: 21)
QVQLQQPGAELVKPGASVKMSCKASGYTFTSYNMHWVKQTPGRGLEWIGAIYPGNGDTSY
NQKFKGKATLTADKSSSTAYMQLSSLTSEDSAVYYCARSTYYGGDWYFNVWGAGTTVTVS (VH)
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKV (CH)
9. anti-Her2Fab light chain-nucleic acid (SEQ ID NO: 2)
GATATTCAGA TGACCCAGAG CCCGAGCAGC CTGTCCGCAA GCGTCGGCGA CCGCGTCACG
ATTACCTGCC GTGCAAGCCA GGATGTCAAC ACCGCAGTGG CTTGGTATCA GCAAAAACCG
GGTAAAGCGC CGAAACTGCT GATTTATAGT GCCTCCTTTC TGTACTCTGG CGTGCCGAGT
CGTTTCTCAG GTTCGCGCAG CGGCACCGAT TTTACCCTGA CGATCAGCTC TCTGCAGCCG
GAAGACTTCG CCACGTATTA CTGCCAGCAA CATTATACCA CGCCGCCGAC CTTTGGTCAA
GGCACGAAAG TCGAAATTAA A (VL)
ACGGTGGCTG CACCATCTGT CTTCATCTTC CCGCCATCTG ATGAGCAGTT GAAATCTGGA
ACTGCCTCTG TTGTGTGCCT GCTGAATAAC TTCTATCCCA GAGAGGCCAA AGTAGAGTGG
AAGGTGGATA ACGCCCTCCA ATCGGGIAAC TCCCAGGAGA GTGTCACAGA GCAGGACAGC
AAGGACAGCA CCTACAGCCT CAGCAGCACC CTGACGCTGA GCAAAGCAGA CTACGAGAAA
CACAAAGTCT ACGCCTGCGA AGTCACCCAT CAGGGCCTGA GCTCGCCCGT CACAAAGAGC
TTCAACAGGG GAGAGTGT (CL)
10. anti-Her2Fab light chain-amino acid (SEQ ID NO: 23)
DIQMTQSPSSLSASVGDPVTITCRASQDVNTAVAWYQQKPGKAPKLLIYSASFLYSGVPS
RFSGSRSGTDFTLTISSLQPEDFATYYCQQHYTTPPTEGQGTKVEIK (VL)
TVAAPSVFIF PPSDEQLKSG TASVVCLLNN FYPREAKVQW KVDNALQSGN SQESVTEQDS
KDSTYSLSST LTLSKADYEK HKVYACEVTH QGLSSPVTKS FNRGEC (CL)
11. anti-Her2Fab heavy chain-nucleic acid (SEQ ID NO: 24)
GAAGTGCAGC TGGTTGAATC TGGCGGTGGC CTGGTTCAAC CGGGTGGCTC CCTGCGTCTG
TCATGTGCAG CATCGGGTTT CAACATTAAA GATACCTATA TCCACTGGGT TCGTCAGGCA
CCGGGTAAAG GCCTGGAATG GGTCGCTCGC ATTTACCCGA CCAATGGTTA TACGCGTTAC
GCGGATAGCG TGAAAGGCCG CTTCACCATC AGCGCAGACA CCTCTAAAAA CACGGCTTAT
CTGCAGATGA ACTCGCTGCG TGCGGAAGAT ACGGCCGTTT ATTACTGTAG CCGCTGGGGT
GGCGACGGCT TTTACGCAAT GGATTACTGG GGTCAAGGCA CGCTGGTCAC GGTCTCA (VH)
GCTAGCACCA AGGGCCCATC GGTCTTCCCC CTGGCACCCT CCTCCAAGAG CACCTCTGGG
GGCACAGCGG CCCTGGGCTG CCTGGTCAAG GACTACTTCC CCGAACCGGT GACGGTGTCG
TGGAACTCAG GCGCCCTGAC CAGCGGCGTG CACACCTTCC CGGCTGTCCT ACAGTCCTCA
GGACTCTACT CCCTCAGCAG CGTGGTGACC GTGCCCTCCA GCAGCTTGGG CACCCAGACC
TACATCTGCA ACGTGAATCA CAAGCCCAGC AACACCAAGG TGGACAAGAA AGTT (CH)
12. anti-Her2Fab heavy chain-amino acid (SEQ ID NO: 25)
EVQLVESGGGLVQGGSLRLSCAASGFNIKDTYIHWVRQAPGKGLEWVARIYPTNGYTRY
ADSVKGRFTISADTSKNTAYLQMNSLRAEDTAVYYCSRWGGDGFYAMDYWGQGTLVTVS (VH)
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKV (CH)

Any of the antibodies or polypeptides described above may further include additional polypeptides, for example, a signal peptide that directs the secretion of the encoded polypeptide, an antibody constant region as described herein, or other heterologous polypeptides as described herein.

Those of ordinary skill in the art will understand that the antibodies disclosed herein can be modified so that they differ in amino acid sequence from the natural binding polypeptides from which they are derived. For example, a polypeptide or amino acid sequence derived from a specific protein may be similar, for example it has a certain percentage of identity to the starting sequence, for example, it may have 60%, 70%, 75%, 80%, 85%, 90%, 95%, 98%, or 99% identity with the starting sequence.

In addition, conservative substitutions, deletions or insertions of nucleotides or amino acids can be made in the “non-essential” amino acid region. For example, a polypeptide or amino acid derived from a specific protein may be the same as the starting sequence, except for one or more single amino acid substitutions, insertions, or deletions (e.g., one, two, three, four, five, six, Seven, eight, nine, ten, fifteen, twenty or more individual amino acid substitutions, insertions or deletions). In certain embodiments, compared to the starting sequence, the polypeptide or amino acid sequence derived from a particular protein has one to five, one to ten, or one to twenty individual amino acid substitutions, insertions, or deletions. In other embodiments, the antigen-binding polypeptides of the present invention may contain conservative amino acid substitutions.

In “conservative amino acid substitutions,” one amino acid residue is replaced by an amino acid residue with a similar side chain. Amino acid residue families with similar side chains have been defined in the art, including basic side chains (such as lysine, arginine, histidine), acidic side chains (such as aspartic acid, glutamic acid), uncharged polar side chains (such as glycine, asparagine, glutamine, serine, threonine, tyrosine, cysteine), non-polar side chains (such as alanine, valine, leucine, isoleucine, proline, phenylalanine, methionine, tryptophan), β branched side chains (such as threonine, valine, isoleucine) and aromatic side chains (such as tyrosine, phenylalanine, tryptophan, histidine). Therefore, non-essential amino acid residues in immunoglobulin polypeptides are more suitable to be replaced by other amino acid residues from the same side chain family. In another embodiment, the amino acid chain may be replaced by a chain that is structurally similar but differs in the order or component of the side chain family members.

The table below provides non-limiting examples of conservative amino acid substitutions. The similarity score of 0 or higher in the table indicates conservative substitutions between two amino acids.

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

In some embodiments, antibodies can be conjugated to therapeutic agents, prodrugs, peptides, proteins, enzymes, viruses, lipids, biological effect modifiers, or pharmaceutical formulations.

These antibodies can be conjugated or fused to therapeutic agents, which may include detectable labels (such as radiolabels), immunomodulators, hormones, enzymes, oligonucleotides, photosensitizing therapeutic agents or diagnostic agents, cytotoxic agents (drugs or toxins), ultrasound enhancers, non-radioactive labels, their combinations, and other agents known in the art.

By coupling to a chemiluminescent compound to label the antibody, the antibody can be detected. Then, by detecting the presence of fluorescence (which occurs during the chemical reaction), the presence of chemiluminescent labeled antigen-binding peptides is determined. Examples of extremely useful chemiluminescent labeling compounds are luminol, isoluminol, theromatic acridinium ester, imidazole, acridinium salt and oxalate ester.

As mentioned above, the bispecific antibodies, vanants or derivatives thereof of the present invention can be used in certain methods of treatment and diagnosis related to cancer or infectious diseases.

### Treatment and Therapy

The present invention further relates to antibody-based therapy, which involves administering the bispecific antibody of the present invention to patients, such as animals, mammals, and humans, to treat one or more diseases or symptoms described herein. The therapeutic composition of the present invention includes, but is not limited to, the antibody of the present invention (including the variants and derivatives thereof described herein) and nucleic acid or polynucleotide (including the variants and derivatives thereof described herein) encoding the antibody of the present invention.

The antibodies of the present invention can be used to treat, inhibit or prevent the following diseases, disorders, or conditions, including, for example, malignant diseases, disorders, or conditions (such as cancers) associated with increasing cell survival or inhibiting cell apoptosis. The cancers include but are not limited to follicular lymphoma, cancers with p53 mutations, and hormone-dependent tumors (including but not limited to colon cancer, heart tumors, pancreatic cancer, melanoma, retinoblastoma, malignant glioma, lung cancer, colorectal cancer, testicular cancer, gastric cancer, neuroblastoma, myxoma, myoma, lymphoma, endothelioma, osteoblastoma, osteoclastoma, osteosarcoma, chondrosarcoma, adenocarcinoma, breast cancer, prostate cancer, Kaposi's sarcoma); autoimmune disorders (such as multiple sclerosis, Sjogren syndrome. Grave disease. Hashimoto's thyroiditis, autoimmune diabetes, biliary cirrhosis, Behcet's disease, Crohn's disease, polymvositis, systemic lupus erythematosus, immune-related glomerulonephritis, autoimmune gastritis, autoimmune thrombocytopenic purpura and rheumatoid arthritis); and viral infections (such as herpes virus, poxvirus and adenovirus), inflammation, graf-versus-host disease (acute and/or chronic), acute graft rejection, and chronic graft rejection. The antigen-binding polypeptides, variants or derivatives thereof of the present invention are used to inhibit the development, evolution and/or metastasis of cancer, especially the cancers listed in the above or subsequent paragraphs.

The antibody of the present invention can also be used to treat infectious diseases caused by microorganisms or kill microorganisms by targeting microorganisms and immune cells to affect the elimination of microorganisms. In one aspect, the microorganism is a virus (including RNA and DNA viruses), gram-positive bacteria, gram-negative bacteria, protozoa, or fungi.

The specific dosage and treatment regimen for any particular patient will depend on many factors, including specific antigen-binding polypeptide, its variants or derivatives used, patient's age, weight, general health, gender, diet, time of administration, excretion rate, combination of drugs, and severity of the specific disease being treated. The physician's judgment on such factors falls within the judgment of those of ordinary skill in the art. The dosage will also be based on the individual patient being treated, route of administration, type of formulation, the characteristics of the composition used, the severity of the disease, and the desired effect. The dosage can be determined by the principles of pharmacology and pharmacokinetics well known in the art.

Methods of administration of the bispecific antibodies and variants include, but are not limited to, intradermal, intramuscular, intraperitoneal, intravenous, subcutaneous, intranasal, epidural and oral routes. The antigen-binding polypeptide or composition can be administered by any convenient route, for example, by infusion or bolus injection, or absorption through epithelial or mucosal protective layer (such as oral mucosa, rectum and intestinal mucosa, etc.); it can be used in combination with other biologically active preparations. Therefore, the pharmaceutical composition containing the antigen-binding polypeptide of the present invention can be administered via oral cavity, rectum, parenteral, vaginal, abdominal cavity, topically (such as by powder, ointment, drops or skin patch), buccal, or oral or nasal cavity.

The term “parenteral” as used herein refers to the mode of administration, which includes intravenous, intramuscular, intraperitoneal, intrasternal, subcutaneous and intraarticular injections and infusions.

Administration can be systemic or local. In addition, it is expected that the antibody of the present invention can be introduced into the central nervous system by any suitable route, including intracerebroventricular and intrathecal injection; intraventricular catheters, for example, connected to a reservoir (such as Ommaya reservoir), can be used to facilitate intraventricular injection. Pulmonary administration can also be used, for example by using inhalers or nebulizers and formulations with aerosols.

It may be desirable to apply the bispecific antibody or composition of the present invention locally to the area in need of treatment; this can be achieved, for example, but not limited to, local infusion during surgery, local application (for example, combined use with wound dressings), injections, catheters, suppositories, or implants (implants are porous, non-porous, non-permeable or gel-like materials, including films (such as silicone membranes) or fibers)). Preferably, when administering a protein (including an antibody) including the antibody of the present invention, care should be taken to use a material that does not adsorb the protein.

The effective amount of the antibody of the present invention in the treatment, inhibition and prevention of inflammation, immune or malignant diseases, disorders or conditions can be determined by standard clinical techniques. In addition, in vitro assays can optionally be used to help identify the optimal dose range. The exact dosage used in the formulation will also depend on the route of administration and the severity of the disease, disorder, or condition, and should be determined based on the judgment of the practitioner and the circumstances of each patient. The effective dose can be deduced from a dose-response curve derived from an in vitro or animal model test system.

As a general suggestion, the dose of the antigen-binding polypeptide of the present invention administered to a patient is usually 0.1 mg/kg to 100 mg/kg patient body weight, 0.1 mg/kg to 20 mg/kg patient body weight, or 1 mg/kg to 10 mg/kg patient body weight. In general, due to the immune response to foreign polypeptides, human antibodies have a longer half-life in the human body than antibodies from other species. Therefore, lower dosing dosage and lower dosing frequency of human antibodies are usually possible. Moreover, the administration frequency and dosage of the antibodies of the present invention can be reduced by modification (such as lipidation) to enhance the uptake of these antibodies and tissue penetration (such as, into the brain).

Methods for the treatment of infections or malignant diseases, disorders or conditions (including administering the antibodies, variants or derivatives of the present invention), before being used in humans, are usually tested in vitro, and then in vivo in acceptable animal models to obtain the desired therapeutic or preventive activity. Suitable animal models (including transgenic animals) are well known to those of ordinary skill in the art. For example, in vitro experiments that demonstrate the therapeutic utility of the antigen-binding polypeptides described herein include the effect assays of the antigen-binding polypeptides on cell lines or patient tissue samples. The effects of the antigen-binding polypeptide on cell lines and/or tissue samples can be determined using techniques known to those skilled in the art, such as tests disclosed elsewhere herein. According to the present invention, in vitro tests that can be used to determine whether a specific antigen-binding polypeptide needs to be used include in vitro cell culture tests in which a patient tissue sample is grown in a culture medium and exposed to or otherwise administered with antibodies and observing the effect of this antibody on the tissue sample.

In a further embodiment, the composition of the present invention is administered in combination with an antitumor agent, antiviral agent, antibacterial agent or antibiotic preparation or antifungal preparation. Any of these formulations known in the art can be administered in the composition of the present invention.

In another embodiment, the composition of the invention is administered in combination with a chemotherapeutic agent. The chemotherapeutic agents that can be administered with the composition of the present invention include, but are not limited to, antibiotic derivatives, such as doxorubicin, bleomycin, daunorubicin, defensin; anti-estrogens such as tamoxifen; antimetabolites such as fluorouracil, 5-FU, methotrexate, fluorouridine, interferon α-2b, glutamic acid, pracamycin, mercaptopurine and 6-mercaptoguanine; cytotoxic agents such as Carmustine, BCNU, lomustine, CCNU, cytarabine, cyclophosphamide, estramustine, hydroxyurea, procarbazine, mitomycin, busulfan, cisplatin and vincristine sulfate; hormones such as medroxyprogesterone, estramustine sodium phosphate, ethinyl estradiol, estradiol, megestrol acetate, medroxyprogesterone, diethylstilbestrol diphosphate, chlorinestrol, and testosterone; nitrogen mustard derivatives, such as chlorambucil, chlorambucil, dichloromethyldiethylamine (chlorambucil), thiotepa; steroids such as betamethasone sodium phosphate; and others, such as dacarbazine, asparaginase, mitotane, vincristine sulfide, vinblastine sulfide, and etoposide.

In another embodiment, the composition of the invention is administered in combination with cytokines. Cytokines that can be administered with the composition of the present invention include but are not limited to IL-2, IL-3, IL-4, IL-5, IL-6, IL-7, IL-10, IL-12, IL-13, IL-15, anti-CD40, CD40L and TNF-α.

In other embodiments, the composition of the invention is administered in combination with other therapeutic or preventive therapies (such as radiation therapy).

### Pharmaceutical Compositions

The invention also provides a pharmaceutical composition. Such a composition includes an effective amount of the antibody and an acceptable carrier. In a specific embodiment, the term “pharmaceutically acceptable” means approved by a regulatory agency of the federal or state government, or listed in the U.S. Pharmacopoeia or other generally recognized pharmacopoeia for use in animals, more specifically for human. Further, the “pharmaceutically acceptable carrier” will usually be a non-toxic solid, semi-solid or liquid filler, diluent, encapsulating material or any type of excipient.

The term “carrier” refers to a diluent, adjuvant, excipient or carrier with which the drug is used. Such pharmaceutical carriers can be sterile liquids, such as water and oils, including oils of petroleum, animal, vegetable, or synthetic origin, such as peanut oil, soybean oil, mineral oil, sesame oil, and the like. When the pharmaceutical composition is administered intravenously, water is the preferred carrier. Salt solutions and aqueous glucose and glycerin solutions can also be used as liquid carriers, especially for injectable solutions. Suitable pharmaceutical excipients include starch, glucose, lactose, sucrose, gelatin, malt, rice, flour, chalk, silica gel, sodium stearate, glyceryl monostearate, talc, sodium chloride, skimmed milk powder, glycerol, propylene, ethylene glycol, water, ethanol, etc.

If desired, the composition may also contain small amounts of wetting or emulsifying agents or pH buffering agents, such as acetate, citrate or phosphate. It can also be expected to add antibacterial agents such as benzyl alcohol or methyl benzoate, antioxidants such as ascorbic acid or sodium bisulfite, chelating agents such as ethylenediaminetetraacetic acid and agents for isotonicity adjustment such as chlorine sodium or dextrose. These compositions can take the form of solutions, suspensions, tablets, pills, capsules, powders, sustained-release formulations and the like. The composition can be formulated as a suppository using customary binders and carriers such as triglycerides. Oral formulations can include standard carriers such as pharmaceutical grades of mannitol, lactose, starch, magnesium stearate, sodium saccharin, cellulose, magnesium carbonate, and the like. Examples of suitable pharmaceutical carriers are described by E. W. Martin in Remington's Pharmaceutical Sciences, incorporated by reference. Such a composition will contain a therapeutically effective amount of the antigen-binding polypeptide (preferably in a purified form) and an appropriate amount of carrier so as to provide the patient with an appropriate mode of administration. This formulation should suit the mode of administration. This parental preparation can be contained in ampoules, disposable syringes or multiple dose vials made of glass or plastic.

In one embodiment, the composition is formulated into a pharmaceutical composition suitable for intravenous administration to humans according to routine procedures. Generally, the composition for intravenous administration is a solution in a sterile isotonic aqueous buffer. If necessary, the composition may also include a solubilizer and a local anesthetic such as lidocaine to relieve pain at the injection site. The components are usually supplied individually or mixed together in unit dosage form, for example, a lyophilized powder or an anhydrous concentrate in a closed container, for example, an ampoule or sachette indicating the number of active agents. When the composition is administered by infusion, it can be dispersed in an infusion bottle containing sterile pharmaceutical grade water or saline. When the composition is administered by injection, an ampoule of sterile water or saline for injection may be provided so that the ingredients can be mixed before administration.

The composition of the present invention can be formulated in a neutral or salt form. Pharmaceutically acceptable salts include salts formed from anions, such as those derived from hydrochloric acid, phosphoric acid, acetic acid, oxalic acid, tartaric acid, etc., and salts formed from cations, such as those derived from sodium, potassium, ammonium, calcium, hydrogen Salts of iron oxide, isopropylamine, triethylamine, ethylhydroxyethylamine, histidine, procaine, etc.

### Examples

### S-Fab Design and Protein Purification

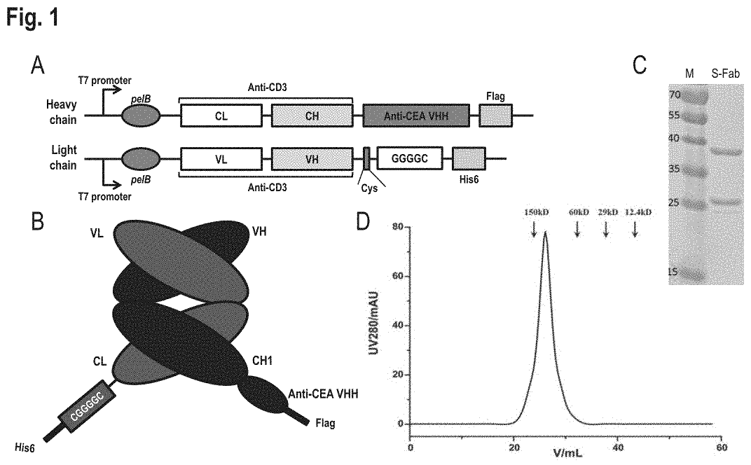

The structure of S-Fab is shown in FIG. 1A. The VH-CH1 and VL-CL fragments of anti-CD3 were synthesized and cloned by standard DNA cloning techniques. A signal sequence pelB was added to the N-terminus for periplasmic expression. For site-specific conjugation of PEG, a cysteine residue was added at the C-terminal of light chain followed by a short linker, and then another cysteine (CGGGGC) and a his6 tag. S-Fab was constructed via the heterodimerization of VL-CL/VH-CH1 (anti-CD3 Fab) with anti-CEA VHH nanobody. Flag-tag was added to the C-terminal of heavy chain for detection (FIG. 1B).

To produce the S-Fab, the two plasmids encoding respective VH—CH—VHH and VL-CL polypeptides were co-transformed into BL21 (DE3, codon plus) competent cells with proper antibiotics. When the absorbance (OD600) of cell culture reached 0.8, 0.2 mM isopropyl-β-d-thiogalactoside (IPTG) was added to induce protein expression. The cells were cultured at 16° C. for another 40 hrs before harvesting. After harvesting the cells by centrifugation, periplasmic extraction was performed by re-suspending the cell pellets 1:4 (g:mL) in a pre-cooled sucrose solution (20 mM Tris-HCl pH 7.5; 25% (w/v) sucrose; 1 mM EDTA). After 15 min incubation on ice, the suspension was centrifuged at 10,000 g for 20 min, and the supernatant fraction was collected as the sucrose fraction. The pellet was then re-suspended in a chilled periplasmic solution (5 mM MgCb) and centrifuged at 10,000 g for 20 min. The supernatant was gathered as the periplasmic fraction.

The S-Fab protein was then purified from the combined sucrose and periplasmic fractions by a two-step purification: first by the immobilized Ni-NTA affinity chromatography (GE Health, USA), and then by an IgG-CH1 affinity matrix (Lot, 194320005; hermoFisher Scientific Inc, USA) (FIG. 1C). Gel filtration analysis was performed using a Bio-Rad FPLP system and GE Superdex 200™ Increase 10/300 GL column with a flow rate of 0.5 ml/min. Fractions (0.5 ml per fraction) were collected, and then subjected to the SDS-PAGE analysis under reducing conditions. The resulting fractions were visualized by the coomassie blue staining. Protein markers (Lot, MWGF200; Sigma Aldrich Co.. Ltd. USA) were loaded as standard controls for gel filtration analysis. Gel filtration analysis showed the intact S-Fab antibodies with molecular weights of ˜130 kD (FIG. 1D), indicated the dimerization of S-Fab (˜65 kD of monomer), likely formed by the disulfide bond(s) of C-terminal cysteine residues of VL-CL.

### Conjugation of S-Fab to PEG (PEGylation)

The S-Fab was engineered with two terminal cysteine residues located at the C-terminal of CL, which served as the sites for conjugation with a 20 kDa linear MAL-PEG-OMe. S-Fab (approximately 1.35 mg/mL (about 20 μM) in 5.0 mL phosphate buffered saline (PBS, pH 7.4) and three molar equivalents of 1 mM tris (2-carboxyethyl) phosphine (TCEP, final 60 μM, approximately 300 μL) were mixed and incubated for two hours at 22° C. to obtain the reduced S-Fab fragments.

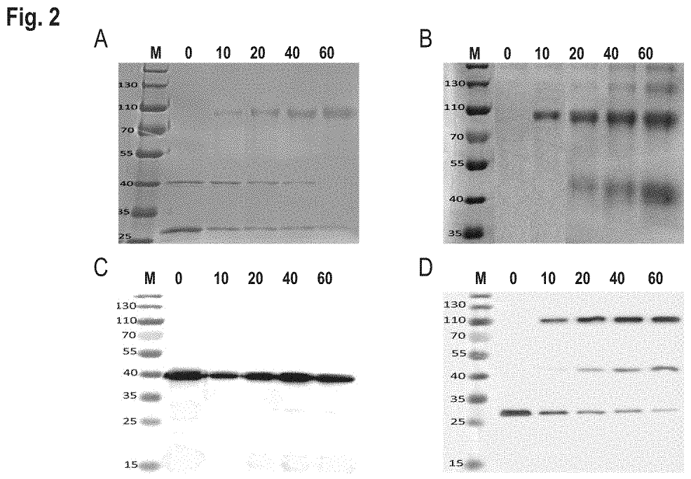

In order to explore the optimal molar ratio of MAL-PEG-OMe and S-Fab in the PEGylation process, we proceeded a series of reactions with the molar equivalent of PEG:S-Fab as 0:1, 10:1, 20:1, 40:1 and 60:1, respectively (FIG. 2A). MAL-PEG-OMe was dissolved in the sterile water to obtain a working concentration of 20 mg/mL (1 mM). The PEGylation of S-Fab was carried out by mixing the MAL-PEG-OMe (at the working concentration) with reduced S-Fab and shaking at 22° C. for 2 hrs. The resulting samples were subjected to 12% SDS-PAGE electrophoresis (5 ul/sample/PAGE), then for coomassie blue and barium iodide staining of PEG according to reference (FIGS. 2A and 2B). After electrophoresis, the western blot assay was used to detect the PEGylated chain. Briefly, another two gels were transferred to polyvinylidene fluoride membrane (Millipore, USA). After two-hour blocking with 5% skimmed milk, membranes were respectively incubated with mouse monoclonal anti-flag HRP (1:2000, for heavy chain) and mouse monoclonal anti-his IgG (1:3000, for light chain) in 5% skimmed milk. The secondary antibody (goat anti-mouse HRP-conjugated IgG, 1:3000) were incubated with the light chain membrane for another one hour after washing with TBST buffer. The membranes were developed with Pierce's West Pico chemiluminescence substrate (Millipore, USA) after washing with TBST buffer. PEGylated S-Fab was purified using an AKTA avant25 FPLC purification system (GE Healthware, USA) and Superdex 10/300 GL column with a flow rate of 0.8 ml/min. The column was first equilibrated with two column volumes (CV) of distilled water and then two CV of PBS before applying samples. All the collected fractions were analyzed by coomassie blue and barium iodide complex dye after SDS-PAGE at reducing conditions. Fractions for the purified PEGylated S-Fab were pooled together for further studies.

Increased ratio of PEG:S-Fab in the PEGylation of S-Fab with MAL-PEG-OMe resulted in a higher molecular weight band (˜107 kD and ˜45 kD), indicating the maleimide functionalized PEG was conjugated to S-Fab. The 45 kD band suggested the single conjugation at the single C-terminal cysteine (FIGS. 2B and 2D). The ˜107 kD and indicates both cysteine residues at the VL-CL terminal were PEGylated (FIGS. 2B and 2D). The ˜107 kD band appeared first and accounts for the majority of light chain, indicating the preferred conjugation on both cysteine residues (FIGS. 2B and 2D). As no decrease of VH—CH-VHH was observed (FIGS. 2A and 2C) and no high molecular weight of VH—CH1-VHH was observed based on western blot (FIG. 2C), suggesting no PEGylation on the VH—CH1-VHH chain. As the band of VL-CL chain was decreased (FIGS. 2A and 2D), and PEGylation corresponds to the VL-CL chain based on anti-his western blot (FIGS. 2B and 2D), it suggested only VL-CL had PEGylation. As the molar ratio of PEG:S-Fab increases, there are also slightly increased higher molecular weight PEGylated bands, suggesting further PEGylation on the other cysteine residues on VH-CH1 or VL-CL. The molar ratio of PEG:S-Fab at 20 was then chosen for conjugation as it had higher VL-CL conjugation without higher molecular weight conjugation.

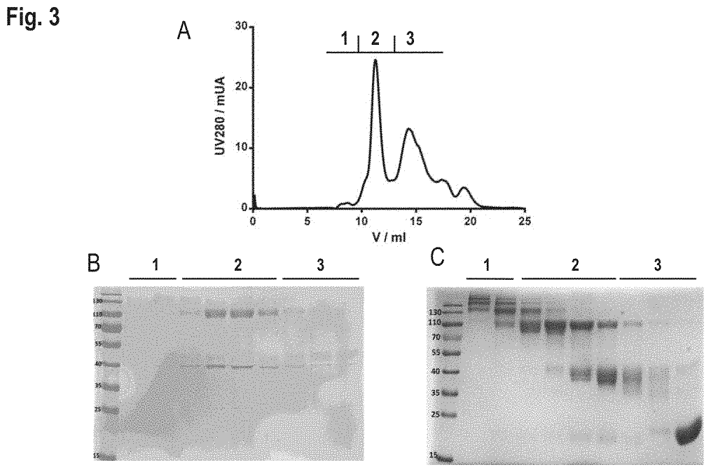

To remove the free PEG, free S-Fab, and high molecular weight proteins, the conjugation reaction mixture was subjected to size exclusion analysis. Based on SDS-PAGE followed by coomassie blue staining (FIG. 3B) and barium iodide staining for PEGylation (FIG. 3C), the high molecular weight fraction 1 (FIG. 3A-3C) contained a large proportion of multiple conjugation species. Fraction 2 (FIG. 3A-3C) contains mostly the double and single site conjugated PEG-S-Fab, while fraction 3 (FIG. 3A-3C) containing free S-Fab, free PEG, and single site conjugated S-Fab. Fractions containing mostly the double and single site conjugated PEG-S-Fab (Fraction 2 in FIG. 3) were pooled for further studies.

PEG-S-Fab can bind the tumor antigen CEA and CD3⁺ T cells.

The bispecific S-Fab has two different binding sites, the anti-CEA VHH recognizing CEA on tumor cells, and the anti-CD3 recognizing CD3′ on T cells. To check whether PEGylation of S-Fab affects the binding of CEA-positive cancer cells, flow cytometry analysis was performed using LS174T cells, which have over-expression of CEA. CEA-negative cell line SKOV3 was used as negative control.

Briefly, 1×106 (for LS174T and SKOV3) or 5×105 (for T cells) cells per sample were collected by centrifugation at 1000 rpm for 5 min and then washed once with 1.0 mL of ice-cold PBS with 0.2% bovine serum albumin (BSA). The primary antibodies, including S-Fab, PEG-S-Fab, and the blank control (Vehicle, PBS only) were added to a final concentration of 10 μg/mL, and then incubated on ice for one hour followed by washing twice with ice-cold PBS with 0.1% BSA. Anti-CD3 FITC (OKT3, final concentration of 10 μg/mL) was used as positive control for CD3⁺ antigen binding analysis. Goat anti-human IgG (H+L)-AlexaFluor 488 antibody was then added to a final concentration of 5 μg/mL. The cells were incubated on ice for another hour. After washing the cells twice, flow cytometric detection was then performed.

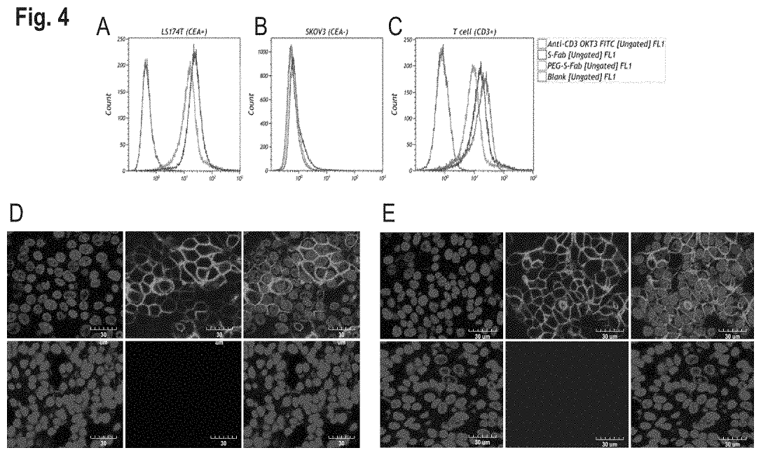

Both S-Fab and PEG-S-Fab showed obviously specific fluorescence intensity shifts, suggesting that PEG-S-Fab can still bind to LS174T cells (FIG. 4A). The slight left shift of PEG-S-Fab to the value of S-Fab (FIG. 4B) indicates that the PEGlyation of S-Fab may slightly reduce the binding affinity of S-Fab to its antigen. For SKOV3 cells, both S-Fab and PEG-S-Fab cannot bind the cells due to the lack of CEA antigen on cell surface, suggesting that PEG-S-Fab retains the specific antigen binding (FIG. 4B). Similarly, for the CD3⁺ on the T cells, the obvious fluorescence intensity shift of S-Fab and PEG-S-Fab was also presented. PEGylation may also slightly decrease the binding activity to the CD3⁺ antigen on T cells (FIG. 4C). The only slightly reduced binding activities of PEG-S-Fab suggested that site specific conjugations in the middle of S-Fab had minimal effects on bispecific antibody binding affinity.

To further analyze the binding of S-Fab and PEG-S-Fab to cell surface CEA, immunofluorescence assay was performed. Briefly. LS174T and SKOV3 cells (2.5×105 cells in 1.0 mL, respectively) were plated on 30-mm confocal glass bottom dishes (NEST, cat. 801002) to 80% confluence. The cells were then washed with cold PBS three times before fixing with 4% paraformaldehyde. The fixed cells were incubated with 20 μg of S-Fab or PEG-S-Fab and then 10 μg goat anti-human IgG (H+L)-AlexaFluor 488 antibody for 2 hour at 4° C. respectively. The cell nuclei were counterstained with DAPI. After washing with PBS, samples were then examined using Olympus FV3000 laser scanning confocal microscope and analyzed by Olympus FV31S-SW_V2.1 software.

Both S-Fab (upper panel in FIG. 4D) and PEG-S-Fab (upper panel in FIG. 4E) can bind CEA positive LS174T cells. For CEA-positive SKOV3 cells, no binding was observed for S-Fab (lower panel in FIG. 4D) or PEG-S-Fab (lower panel in FIG. 4E), further supporting that PEGlyation maintains the specific binding affinity of S-Fab to its antigen.

### PEG-S-Fab has Potent Specific Cytotoxicity Against Tumor Cells

The CEA-positive human LS174T cells and the CEA-negative human SKOV3 cells were used to assess the in-vitro growth inhibitory effects of S-Fab and PEG-S-Fab. Briefly, LS174T and SKOV3 cells were used as target cells (T), and freshly prepared human CD3⁺ T cells without prior stimulation were used as effector cells (E). In vitro cytotoxicity assays were performed in 96-well microplates in triplicates by seeding 5,000 target cells per well in 100 μL of corresponding media. After a 6-hour incubation, an equal volume of CD3⁺ T cells were added to each well at an E:T ratio of 10:1, and a series of concentrations (0.033, 0.1, 0.33, 1, 3.3, 10, 33 and 100 nM) of S-Fab or PEG-S-Fab were then added correspondingly. After 72-hour incubation, cell viability was evaluated via a CCK8 assay according to the manufacturer protocol. The absorbance values were detected using a TECAN microplate reader at 450 nm. The survival rate (100%) was calculated as: [(As−Ab)(A0−Ab)]×100%, where As is the absorbent value of the measurement group, Ab is the absorbent value of medium and A0 is the absorbent value of measurement group at 0 nM.

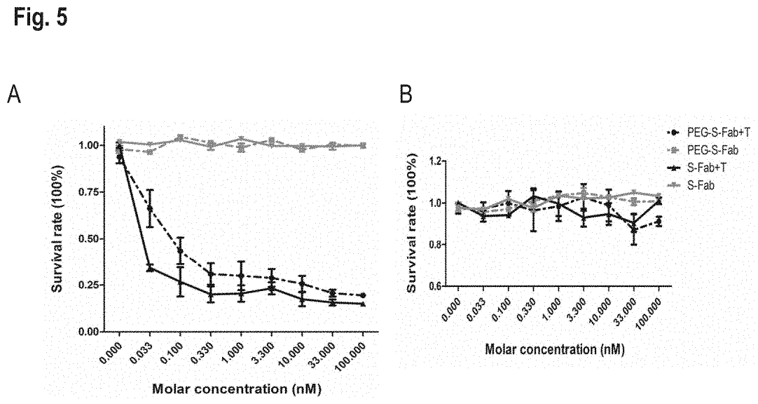

Both S-Fab and PEG-S-Fab can kill CEA-positive LS174T cells efficiently even at 0.033 nM (FIG. 5A) while no cytotoxcity against CEA-negative SKOV3 cells (FIG. 5B). The PEG-S-Fab has slightly decreased cytotoxicity than S-Fab (FIG. 5A). This could be due to the slightly decreased antigen binding or steric interference of PEGylation. However, the level of decreased activity seems much lower than many other PEGlyated proteins.

### PEGylation Prolongs In Vivo Half-Life of S-Fab

To determine the circulating half-life of PEG-S-Fab, the intravenous PK profiles of S-Fab and PEG-S-Fab in rat were analyzed. SPF male SD rats (250-300 g) were used for the PK assay. Food was controlled to maintain animals below 350 g in weight. S-Fab (1.0 mg/kg), PEG-S-Fab (1.0 mg/kg) or a volume equivalent of vehicle solution PBS was administered through the caudal vein. The blood sample (each approximately 150-200 μL) was taken from the orbital vein using a capillary under isoflurane anesthesia at 0, 0.5, 1, 2, 4, 8, 16, 24, 36, 48, 72, 96 and 144 hrs after administration. All blood samples were collected into heparinized tubes. Plasma was obtained via centrifugation at 3,500 g for 30 min, and then stored at −80° C. until further analysis.

S-Fab and PEG-S-Fab in the plasma samples were quantified using an ELISA assay. Briefly, a 100 μL aliquot of 6D6 (mouse anti-human IgG Fab antibody) (1.0 μg/mL in PBS) was coated to each well of a 96-well ELISA microplate (ThermoFisher, USA) for 2 hrs at 37° C. The wells were then washed twice with 200 μL of PBST (PBS+0.05% Tween-20). Wells were then blocked with 200 μL of blocking buffer (PBST containing 1% bovine serum albumin) for 2 hrs at 37° C. Each well was then washed five times with PBST prior to the addition of 100 μL of samples or standards. Samples and standards (100, 80, 50, 40, 30, 20, 10, 5, 1 and 0.1 μg/mL) were prepared in the blocking buffer with the standards (S-Fab) prepared in a 1:10 dilution of plasma using PBS, which was important to avoid matrix effects in the assay. For plasma samples, a 1:3 dilution was used. 100 μL sample or standard aliquots was then added in triplicate and incubated at 37° C. for one hour. Each well was washed again with PBST before the addition of 100 μL of secondary antibody (mouse monoclonal anti-flag mzperoxidase (HRP) antibody at 1:500 dilutions) per well at 37° C. for one hour. After washing for five times, a 100 μL aliquot of TMB substrate solution was added to each well. After 10 min incubation, 100 μL of 2M H₂SO₄ was added to stop the reaction. The absorbance was then detected at 450 nm using a TECAN ELISA microplate reader. Serum elimination t_{1/2} and clearance were calculated by 3p97 pharmacokinetic software using standard formula. The results are expressed as the mean±SEM, and comparisons between the groups were made with an unpaired Student's t-test. Differences were considered to be statistically significant if p<0.05.

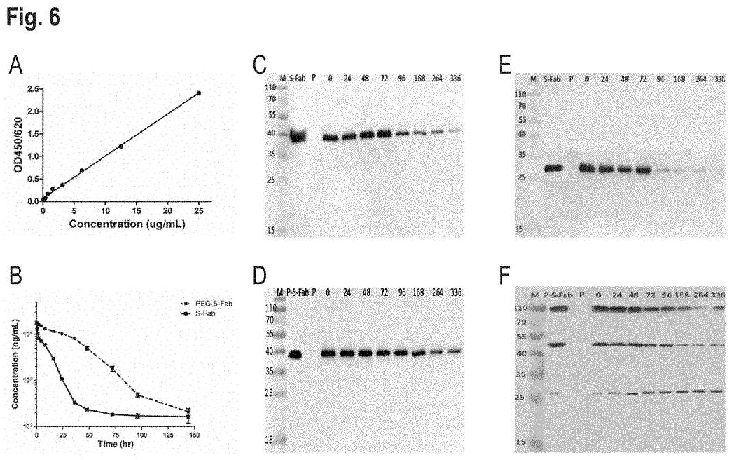

S-Fab and PEG-S-Fab were quantified using the ELISA method as described in the part of 210. Quantification of a series of standards showed that the standard curve was y=0.0934×+0.0748 (0≤x≤25 μg/mL) with R² value of 0.9982 (FIG. 6A). Based on serum protein concentration up to 144 hrs, the S-Fab had an in-vivo half-life of approximately 3.0 hrs, similar to reported half-life of other Fab in vivo [34-36]. PEGylation significantly extended the half-life of PEG-S-Fab to 36.0 hrs, a 12-fold increase to S-Fab (FIG. 6B).

S-Fab and PEG-S-Fab stability was assessed in human fresh plasma over two weeks. Briefly. S-Fab and PEG-S-Fab were diluted with human fresh plasma (without platelet), which generated an initial concentration of 100 μg/mL. The samples were incubated at 37° C. for two weeks. At the time intervals of 0, 24, 48, 72, 96, 168, 264 and 336 hr, 40 μL samples were collected and then stored directly at −80° C. till further analysis. The samples were thawed on ice and then centrifuged at 14,000 rpm for 10 mins at 4° C. The supernatant was then subjected to electrophoresis on 12% SDS-PAGE (5 ul sample per well). After electrophoresis, western blot as descripted in 2.4 was performed to analyze the protein level. When incubated with human plasma in vitro at 37° C., the level of S-Fab decreased sharply after 72 hrs (FIGS. 6C and 6E). For PEG-S-Fab, the decrease was much slower (FIGS. 6D and 6F). Thus. PEGylation can also improve the stability of S-Fab in plasma probably due to the protection of enzymatic digestion in plasma.

### PEG-S-Fab Induces More Potent In Vivo Anti-Tumor Activity

The increased in vivo half-life and comparable in vitro cytotoxicity of PEG-S-Fab prompted us to assess the in vivo anti-tumor activity of PEG-S-Fab in an adoptive xenograft model. The in vivo anti-tumor activities of S-Fab and PEG-S-Fab were studied using NOD/SCID mice engrafted subcutaneously with LS174T cells. Briefly, LS174T cells were harvested and washed once with PBS, and then mixed with human PBMCs freshly isolated from healthy donors. The mixtures of 1×10⁶ LS174T cells and 5×10⁶ human PBMCs were injected subcutaneously into the right flank of NOD/SCID mice in a total volume of 0.2 mL per mouse. One hour after the engraftment, 0.3 nmol S-Fab (20.0 μg per mouse), and 0.3 nmol PEG-S-Fab (32.0 μg per mouse) or the vehicle control (PBS) were injected intraperitoneally. The animals were then treated daily (0.3 nM per mouse in each group) over the following six days. Tumor volume was measured with calipers in two perpendicular dimensions and was calculated using the formula (width²×length)/2. All data were expressed as the mean SE for each group, and differences between groups were determined by two-way ANOVA using GraphPad Prism 5 software.

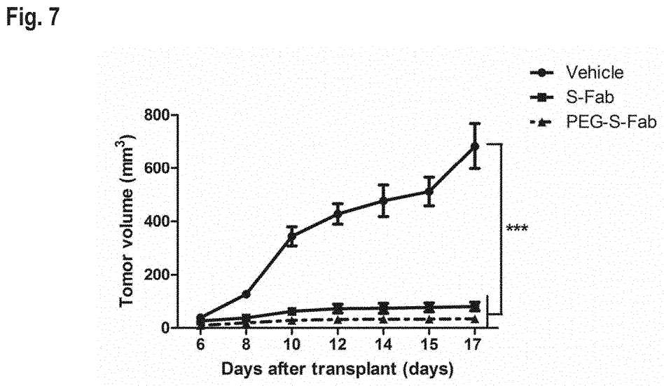

When NOD/SCID mice were transplanted with LS174T cells and fresh isolated human PBMCs, rapid tumor growth was observed. Compared with the vehicle group, significant tumor growth inhibition (p<0.01) was observed when the mice were treated with either S-Fab or PEG-S-Fab in the presence of human PBMCs (FIG. 7). The tumor growth inhibition was more pronounced in the presence of PEG-S-Fab compared to S-Fab (p<0.01), indicating PEGylation of S-Fab protein can enhance the therapeutic efficacy of the S-Fab in the xenograf mouse model.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

When appropriate, the explanatory description herein may be implemented in the absence of any one or more elements, one or more restrictions that are not specifically disclosed herein. Therefore, for example, in each case herein, any one of the terms “including”, “substantially consisting of” and “consisting of” can be replaced by the other two terms. Therefore, it should be understood that although the present invention has been specifically disclosed through preferred embodiments and optional features, those skilled in the art can make modifications and changes to the ideas disclosed herein, and these modifications and changes are still within the scope of the present invention as defined in the appended claims.

1. A PEGylated bispecific antibody, comprising
(a) an antigen-binding fragment (Fab) having a light chain variable region (VL) and a light chain constant region (CL), a heavy chain variable region (VH) and a part of a heavy chain constant region (CH1);
(b) a single domain antigen-binding fragment (VHH) fused to a C-terminus of the part of the heavy chain constant region (CH1) of the antigen binding fragment (Fab); and
(c) polyethylene glycol (PEG) fused to a C-terminus of the light chain constant region (CL) of the antigen-binding fragment (Fab).

2. The PEGylated bispecific antibody of claim 1, wherein the polyethylene glycol is connected to the C-terminus of the light chain constant region (CL) through at least one cysteine residue at the C-terminus of the light chain constant region (CL).

3. The PEGylated bispecific antibody of claim 2, wherein when the polyethylene glycol is connected to the C-terminus of the light chain constant region (CL) through two cysteine residues at the C-terminus of the light chain constant region (CL), a linker sequence is spaced between the cystine residues.

4. The PEGylated bispecific antibody of claim 1, wherein the molecular weight of the polyethylene glycol is 10,000 to 30,000.

5. The PEGylated bispecific antibody of claim 4, wherein the molecular weight of the polyethylene glycol is 20,000.

6. The PEGylated bispecific antibody of claim 1, wherein the antigen-binding fragment is specific for tumor antigens, and the single domain antigen-binding fragment is specific for immune cells.

7. The PEGylated bispecific antibody of claim 6, wherein the tumor antigen is selected from a group consisting of CEA, EGFR, Her2, EpCAM, CD20, CD30, CD33, CD47, CD52, CD133, CEA, gpA33, Mucin, TAG-72, CIX, PSMA, folate binding protein, GD2, GD3, GM2, VEGF, VEGFR, integrin, αVβ3, α5β1, ERBB2, ERBB3, MET, IGF1R, EPHA3, TRAILR1 TRAILR2, RANKL, FAP and Tenascin.

8. The PEGylated bispecific antibody of claim 7, wherein the tumor antigen is CEA or Her2.

9. The PEGylated bispecific antibody of claim 6, wherein the single domain antigen-binding fragment is specific for mammalian T cells or mammalian NK cells.

10. The PEGylated bispecific antibody of claim 6, wherein the single domain antigen binding fragment has specificity for any antigen selected from the group consisting of CD3, CD16, CD19, CD28 and CD64.

11. The PEGylated bispecific antibody of claim 10, wherein the antigen is CD16 or CD3.

12. An engineered bispecific antibody comprising
(a) an antigen-binding fragment (Fab) having a light chain variable region (VL) and a light chain constant region (CL), a heavy chain variable region (VH) and a part of a heavy chain constant region (CH1); and
(b) a single domain antigen binding fragment (VHH) fused to the C-terminus of the part of the heavy chain constant region (CH1) of the antigen binding fragment (Fab);
wherein the C-terminus of the light chain constant region (CL) of the antigen-binding fragment (Fab) is engineered to have one or two cysteine residues.

13. The engineered bispecific antibody of claim 12, wherein when there are two cysteine residues, there is a linker sequence between the two cysteine residues.

14. (canceled)

15. A pharmaceutical composition comprising the bispecific antibody of claim 1 and a pharmaceutically acceptable carrier.
